(19) United States
(12) Patent Application Publication
(10) Pub. No.: US 2021/0050613 A1
(43) Pub. Date: Feb. 18, 2021

(54) NANOSTRUCTURED BIOELECTRODE FOR GLUCOSE OXIDATION, FROM ELECTROGENERATED AROMATIC COMPOUNDS

(71) Applicants: CENTRE NATIONAL DE LA RECHERCHE SCIENTIFIQUE, Paris (FR); UNIVERSITÉ GRENOBLE ALPES, Saint-Martin-d'Héres (FR)

(72) Inventors: Michaël HOLZINGER, Saint-Laurent du Pont (FR); Pierre-Yves BLANCHARD, Montpellier (FR); Alan LE GOFF, Crêts-en-Belledonne (FR); Serge COSNIER, Crolles (FR)

(21) Appl. No.: 16/981,214

(22) PCT Filed: Mar. 15, 2019
(86) PCT No.: PCT/EP2019/056628

§ 371 Date: Sep. 15, 2020

(30) Foreign Application Priority Data
Mar. 16, 2018 (FR) 18/52284

(51) Int. Cl. H01M 8/16; C12N 9/04; C12Q 1/00; G01N 27/327

(52) U.S. Cl. CPC H01M 8/16; C12N 9/0006; B82Y 40/00; G01N 27/3271; C12Y 101/9901; C12Q 1/006

(57) ABSTRACT

The invention relates to a bioelectrode comprising a conductive material, on the surface of which are deposited carbon nanotubes, a redox mediator based on pyrene or a derivative thereof, oxidized in-situ, and an enzyme capable of catalyzing the glucose oxidation. The invention also relates to a process for producing such a bioelectrode, and to the uses thereof.

### FIELD OF THE INVENTION

The present invention relates to a nanostructured bioelectrode from electrogenerated aromatic compounds as well as to the use of these electrogenerated aromatic compounds as a particularly suitable mediator for the transfer of electrons between an enzyme catalyzing the glucose oxidation such as flavin adenine dinucleotide-dependent glucose dehydrogenase (FAD-GDH) and an electrode.

### PRIOR ART

The development of biofuel cells using enzymes is widely described in the literature (cf. U52002/0025469 and EP2375481 A1). These enzymatic fuel cells use biocatalysts, called enzymes, to carry out an oxidation reaction of a fuel (H₂, alcohols, glucose, etc.) at the anode and the reduction of an oxidant (mostly O₂) at the cathode.

The advantage of using enzymes for energy production is their high selectivity towards the substrate. The enzyme FAD-GDH exhibits the properties of oxidizing glucose to gluconic acid. This enzyme has an active site inside its structure: direct electron transfer is therefore impossible and it is necessary to use a redox mediator in order to transfer the electrons from the enzyme to the electrode. Several approaches are described for the immobilization of the redox mediator within the electrode such as encapsulation, polymerization, and covalent grafting. These studies generally show the immobilization of the mediator in an already active state.

Barathi et al. (Barathi, P.; Senthil Kumar, A. Electrochemical Conversion of Unreactive Pyrene to Highly Redox Active 1,2-Quinone Derivatives on a Carbon Nanotube-Modified Gold Electrode Surface and Its Selective Hydrogen Peroxide Sensing. Langmuir 2013, 29 (34), 10617-10623) describe a method of degrading pyrene to an active quinone derivative. The compound is deposited as inactive pyrene on the electrode and then activated (oxidized) by electrochemistry. Barathi et al., also describe the use of such an electrode on which is also deposited cytochrome C and copper (Cu²⁺). This electrode is used as a cathode for the reduction of hydrogen peroxide.

Redox mediators must meet several criteria. The electron transfer must be rapid so as not to limit the catalytic process. The redox mediator must be only slightly, or not at all, released in solution. For this, the molecules used in this study are aromatic molecules because they produce very good interactions with carbon nanotubes. The molecules are physisorbed by π-π interactions. The redox potential of the probe must be greater than the redox potential of the active site of the enzyme but close enough (>50 mV) in order to obtain a high electromotive force (emf=potential of the cathode−potential of the anode) in the biofuel cells.

### DESCRIPTION OF THE INVENTION

The present invention relates to the identification and use of a redox mediator particularly suitable for the production of a bioanode for an enzymatic biofuel cell, in particular an enzymatic biofuel cell comprising a FAD-GDH. This mediator exhibits much better stability over time compared to the redox mediators conventionally used such as 1,4-naphthoquinone.

One aspect of the invention is thus a bioelectrode comprising a conductive material, on the surface of which are deposited carbon nanotubes, a redox mediator based on pyrene, or a derivative thereof, oxidized in-situ and an enzyme capable of catalyzing the glucose oxidation. It should be noted that the term “glucose” used here refers in particular to the D-(+)-enantiomer of glucose (dextrose) which occurs naturally in living organisms.

The electrode according to the invention is preferably a multilayer electrode and advantageously comprises a layer of carbon nanotubes, a layer of pyrene oxidized in-situ and an enzyme layer capable of catalyzing the glucose oxidation. The layers can be deposited successively on a conductive material, which can constitute the carrier for these layers or be itself deposited on an inert carrier. The conductive material can be glassy carbon, pyrolytic graphite (in particular HOPG “highly ordered pyrolytic graphite”) gold, platinum and/or indium tin oxide. Preferably, the material is glassy carbon or pyrolytic graphite.

The use of carbon nanotubes allows the increase of the specific surface of the electrode (porosity) and the formation of a 3D nanostructured network and also makes it possible to ensure the conductivity within the material with its conjugated π system which allows strong non-covalent interaction with oligomer aromatic rings. The ratio of the specific surface area to the number of redox molecules is relatively high and allows the absence of passivation during the electrosynthesis step. It is conventionally accepted that the electropolymerization of organic molecules on electrodes induces passivation resulting in a reduction in the rate of electron transfer. The porous electrodes based on carbon nanotubes (CNT) are produced using commercial, preferably non-functionalized, multi-wall CNTs. Several production methods are suitable, and make it possible to form, among other things, either sheets (buckypapers), pellets, or deposits on the conductive carrier material. The conductive material on which the carbon nanotubes are deposited can also form part of a microporous gas diffusion electrode comprising a GDL (GDL=gas diffusion layer), which layer generally comprises carbon fibers.

Carbon nanotubes are fullerenes composed of one or more sheets of carbon atoms coiled on themselves forming a tube. The tube may or may not be closed at its ends by a hemisphere. Single-sheet carbon nanotubes (SWNT or SWCNT, for single-walled (carbon) nanotubes) and/or multi-sheet carbon nanotubes (MWNT or MWCNT, for multi-walled (carbon) nanotubes) can be used, although multi-sheet carbon nanotubes are preferred.

The combination of the conductive material and carbon nanotubes, which can advantageously be deposited on said material in the form of a layer, makes it possible to obtain a porous carrier capable of receiving the enzyme and its particular mediator (pyrene oxidized in-situ).

When oxidized in-situ, pyrene and the derivatives thereof form particularly effective mediators, in particular of the FAD-GDH enzyme. In particular, the term “pyrene derivative” denotes a molecule included in the group consisting of pyrene where at least one hydrogen atom present on the aromatic polycyclic carbon structure of pyrene is substituted by at least one C2-C22 alkyl group, and in particular a C2-C4 alkyl group, such as ethyl, propyl or butyl.

It is also advantageous to avoid using pyrene derivatives comprising amine or hydroxyl groups, or else halogen atoms.

Pyrene, or a derivative thereof, oxidized in-situ is an organic compound from pyrene or a derivative thereof, which has been deposited on the electrode. Once deposited on the surface of the electrode, the oxidation of pyrene or a derivative thereof can be carried out either by cyclic voltammetry or by chronoamperometry. The compounds formed are one or more type(s) of electroactive quinoic oxide(s). Pyrene, or a derivative thereof, oxidized in-situ acts as a redox mediator of the enzyme which is part of the bioelectrode according to the invention.

The mediator obtained in-situ can in particular be obtained by chronoamperometry and comprise the application to pyrene, or to a derivative thereof, deposited in-situ on the surface of the electrode, of a potential of 1 V at said electrode for a given time, preferably ranging from 10 seconds to 3 minutes, advantageously ranging from 30 seconds to 3 minutes.

Alternatively or in combination, pyrene, or a derivative thereof, oxidized in-situ can be obtained by cyclic voltammetry and comprise the application to pyrene, or to a derivative thereof, deposited in-situ on the surface of the electrode, of a potential varying cyclically from −0.4 V to 1 V. Preferably, the number of cycles applied during this step varies from 3 to 20.

The application of such a signal causes the formation of ketone bonds on the aromatic compound. The mechanism of formation is not completely resolved and therefore the exact nature of the compound formed differs according to the various efforts in the literature, in particular regarding the number of ketone functions created, However, all are in agreement on the quinoic nature of the reaction products.

The enzyme capable of catalyzing the glucose oxidation is preferably a glucose dehydrogenase (GDH) catalyzing the reaction:

D-glucose+acceptor→D-glucono-1,5-lactone+reduced acceptor. The acceptor, or cofactor, is usually an NAD⁺/NADP⁺ or a flavin coenzyme, such as FAD (flavin adenine dinucleotide), or FMN (flavin mononucleotide) which is linked to GDH. A particularly preferred glucose dehydrogenase is flavin adenine dinucleotide-dependent glucose dehydrogenase (FAD-GDH) (EC 1.1.5.9). The term “FAD-GDH” extends to native proteins and their derivatives, mutants and/or functional equivalents. This term extends in particular to proteins which do not differ substantially in structure and/or in enzymatic activity. Thus, it is possible to use for the electrode according to the invention, in combination with a cofactor, an enzymatic protein GDH exhibiting an amino acid sequence having at least 75%, preferably 95%, and particularly preferably 99% identity with the GDH sequence(s) as listed in databases (for example SWISS PROT). A FAD-GDH from Aspergillus sp. is particularly preferred and effective, but other FAD-GDHs from Glomerella cingulata (GcGDH), or a recombinant form expressed in Pichia pastoris (rGcGDH), could also be used.

It is also possible to produce an electrode according to the invention using an oxidoreductase enzyme (EC 1.1.3.4) of the glucose oxidase type (GOx, GOD) which catalyzes the oxidation of glucose to hydrogen peroxide and to D-glucono-δ-lactone. This enzyme, which is a reference enzyme for glucose cells, is also linked to a cofactor such as FAD (flavin adenine dinucleotide). A particularly preferred glucose oxidase is flavin adenine dinucleotide-dependent glucose oxidase (FAD-GOx). This term extends to native proteins and their derivatives, mutants and/or functional equivalents. The term “FAD-GOx” extends in particular to proteins which do not differ substantially in structure and/or in enzymatic activity. Thus, it is possible to use for the electrode according to the invention, in combination with a cofactor, an enzymatic protein GOx exhibiting an amino acid sequence having at least 75%, preferably 95%, and particularly preferably 99% identity with the GOx sequence(s) as listed in databases (for example SWISS PROT). FAD-GOx extracted from Aspergillus niger is particularly preferred.

FAD-GDH exhibits higher activity than glucose oxidase and therefore a higher catalytic current, This is of considerable interest in order to increase the powers generated in enzymatic biofuel cells. It should be noted that unlike glucose oxidase, the FAD-GDH enzyme does not produce hydrogen peroxide. As a result of its oxidizing properties, hydrogen peroxide can present drawbacks for the stability of biofuel cells (membrane, stability of enzymes at the cathode, etc.).

Another aspect of the invention relates to a process for producing a bioelectrode capable of glucose oxidation, said method comprising:

a) a step involving the oxidation of pyrene, or a derivative thereof, wherein said pyrene or said derivative is pre-deposited on the surface of a conductive material, a conductive material on the surface of which carbon nanotubes are also deposited, and

b) a step, preferably subsequent to step a), involving depositing an enzyme capable of catalyzing the glucose oxidation on the surface of said electrode.

The structural characteristics of the bioelectrode according to the process of the invention are advantageous as described above.

According to a preferred aspect of the method according to the invention, the nanotubes are deposited on the conductive material by a so-called dropcasting step.

According to this method, a homogeneous solution or dispersion of a product is deposited on a carrier, then a solvent evaporation step is carried out which allows a thin layer of said product to be deposited on said carrier. Usually the solvent is an organic solvent except for the enzyme.

Thus, for the deposition carbon nanotubes, the solvent selected may be N-methyl-2-pyrrolidone (NMP). The concentration of the solution/dispersion of nanotubes can vary from 1 to 10 mg·mL⁻¹, preferably around 5 mg·mL⁻¹. According to one aspect of the process, the electrode of conductive material is oriented vertically during the deposition of the nanotubes.

According to another preferred aspect of the invention, the pyrene oxidation step is carried out by chronoamperometry and may comprise the application of a potential of 1 V at said surface for a given time, preferably ranging from 10 seconds to 3 minutes, advantageously from 30 seconds to 3 minutes.

Alternatively, or in combination, the pyrene oxidation step is performed by cyclic voltammetry and may comprise the application of a potential varying cyclically from −0.4 V to 1 V at the electrode surface. Preferably_{;} the number of cycles applied varies from 3 to 20 for a scan rate of 100 mV·s⁻¹.

The electrolyte solution which can be used for the chronoamperometry and/or cyclic voltammetry step can be a buffer solution, for example a phosphate buffer solution. The pH of the electrolyte solution is generally 6.5 to 7.5, preferably around 7, because of the optimal enzyme activity around 7.

According to a preferred aspect of the process according to the invention, the pyrene is deposited on a surface of the electrode comprising carbon nanotubes also using a dropcasting step. In this case, the solvent is advantageously dichloromethane, The concentration of the solution can be selected from 5 to 15 mM, in particular around 10 mM.

According to a preferred aspect of the process according to the invention, the enzyme used is a flavin adenine dinucleotide-dependent glucose dehydrogenase or a flavin adenine dinucleotide-dependent glucose oxidase, as described above.

According to another preferred aspect of the process according to the invention, the step of depositing the enzyme on the surface of the bioelectrode is also carried out using a dropcasting step. In this case, the solvent is advantageously an aqueous solution, preferably buffered at pH 7. The concentration of the solution may be from 1 to 10 mg·mL⁻¹, preferably 5 mg·mL⁻¹. The deposition and/or evaporation of the solvent can advantageously take place at atmospheric pressure and room temperature. The drying time is generally selected from 2 to 4 hours.

Obviously, the invention also relates to a bioelectrode obtained directly by the process according to the invention as described above as well as in the implementation examples below. The invention also relates to the applications and uses of such an electrode in various technologies. For example, the invention also relates to the use of a bioelectrode according to the invention as a bioanode suitable for the production of a biofuel cell. Such a biofuel cell is advantageously an enzymatic biofuel cell. Such a biofuel cell comprises, in association with at least one electrode according to the invention, a biocathode. This biocathode can, for example, comprise an enzyme which makes it possible to reduce oxygen, for example based on bilirubin oxidase or laccase. It can comprise, as conductive material, a material of the type as described above and advantageously carbon nanotubes modified with a protoporphyrin allowing direct electron transfer with bilirubin oxidase. If the enzyme is laccase, these carbon nanotubes are advantageously modified with a hydrophobic group such as adamantane, anthracene or pyrene. Mediated electron transfer can also be obtained from MWCNT and the ABTS molecule for both enzymes.

Another use of the electrode according to the invention relates to its use in a glucose biosensor.

Finally, the invention also relates to the use of a pyrene derivative as described above, instead of or in combination with pyrene. The substituted pyrene derivative can also be oxidized in situ using the steps described above and electrodes, cells and glucose biosensors, as well as the processes for producing them are also an object of the invention.

An embodiment of the invention is given by way of non-limiting example and which includes appended drawings which show the following:

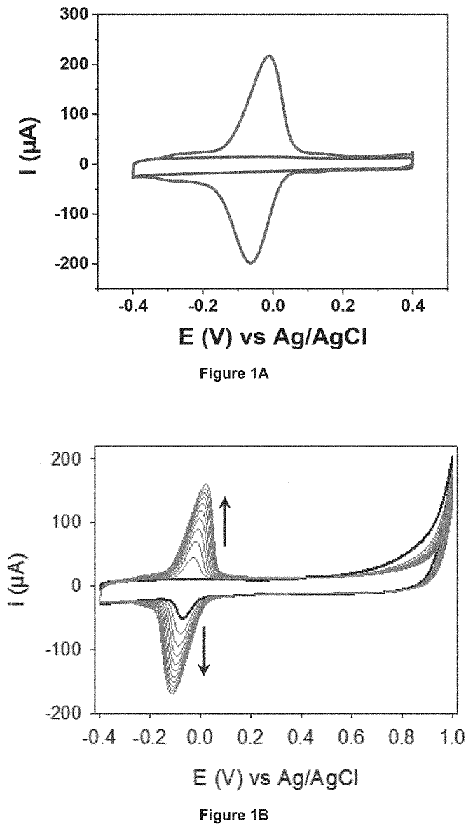

FIG. 1: (A) Voltammograms of a glassy carbon/MWCNT/pyrene electrode before (black) and after chronoamperometry of 1 V vs, Ag/AgCl for 30 seconds (phosphate buffer 0.2 M pH=7)

(B) Electrochemical response of the electrosynthesis of a glassy carbon/MWCNT/pyrene electrode (black=1st cycle/gray=cycles 2 to 6)

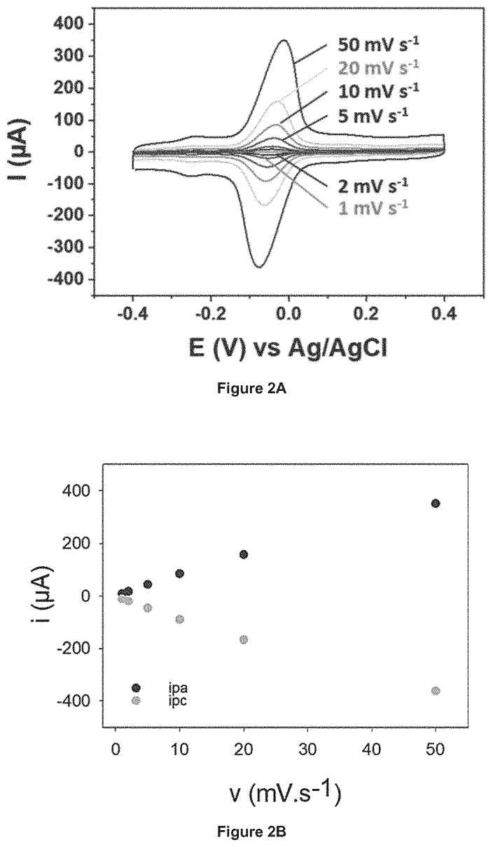

FIG. 2: (A) Voltammograms of a glassy carbon/MWCNT/pyrene redox electrode at different scan rates (phosphate buffer 0.2 M pH=7).

(B) Represents the intensities of the anode and cathode peaks of a glassy carbon/MWCNT/pyrene redox electrode as a function of the scan rate.

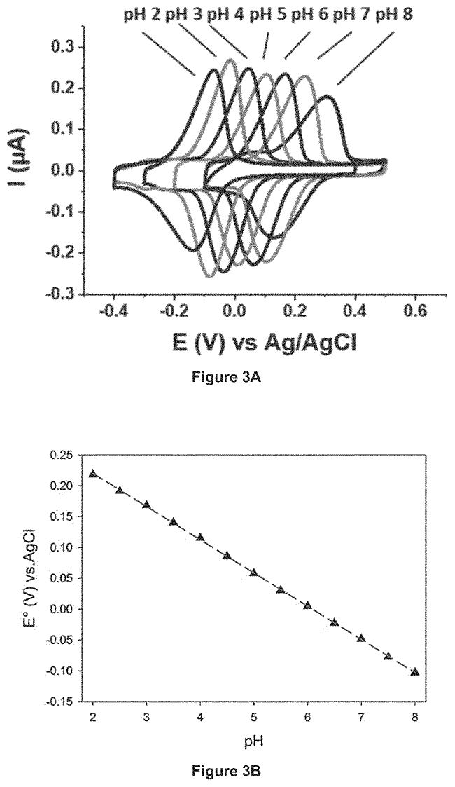

FIG. 3: (A) Voltammograms of a glassy carbon/MWCNT/pyrene redox electrode at different pH values (2, 3, 4, 5, 6, 7, 8)

(B) Representation of the evolution of the standard potential as a function of the pH of a glassy carbon/MWCNT/pyrene redox electrode

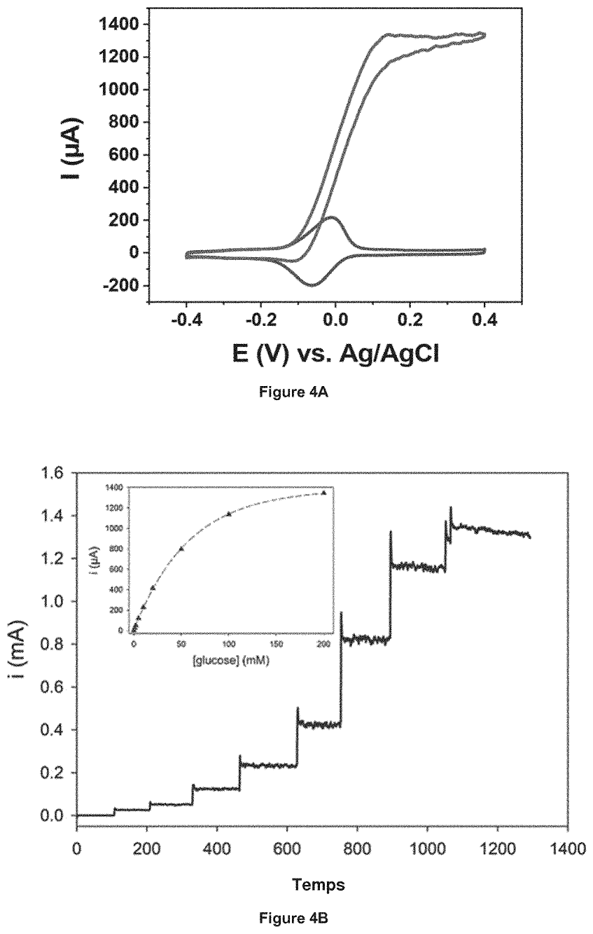

FIG. 4: (A) Electrochemical response of the modified MWCNT/pyrene redox/FAD-GDH electrode in the absence (black curve) and in the presence of 200 mM glucose (gray curve)

(B) Chronoamperometry at 0.2 V vs. Ag/AgCl of the modified MWCNT/pyrene redox/FAD-GDH electrode during glucose injection (1, 2, 5, 10, 20, 50, 100, 200 mM glucose) (cf. insert) Representation of the evolution of the catalytic current as a function of the glucose concentration obtained during chronoamperometry at 0.2 V vs. Ag/AgCl

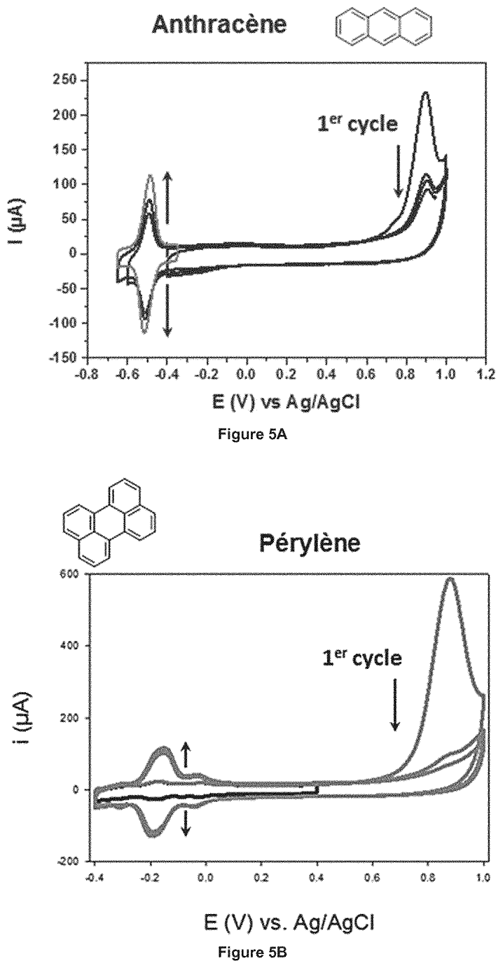

FIG. 5: (A) Electrochemical response of the electrosynthesis of a glassy carbon/MWCNT/anthracene electrode

(B) Electrochemical response of the electrosynthesis of a glassy carbon/MWCNT/perylene electrode

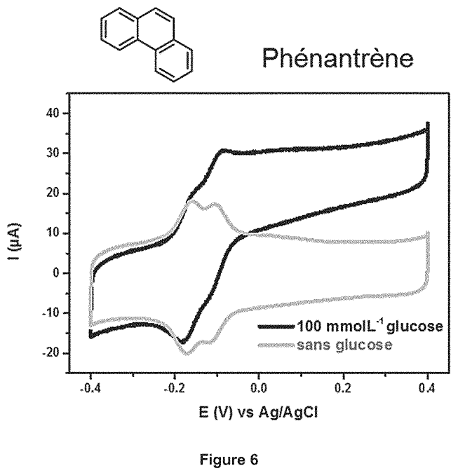

FIG. 6: Electrochemical response of the modified MWCNT/phenanthene redox/FAD-GDH electrode in the absence (black curve) and in the presence of 200 mM glucose (gray).

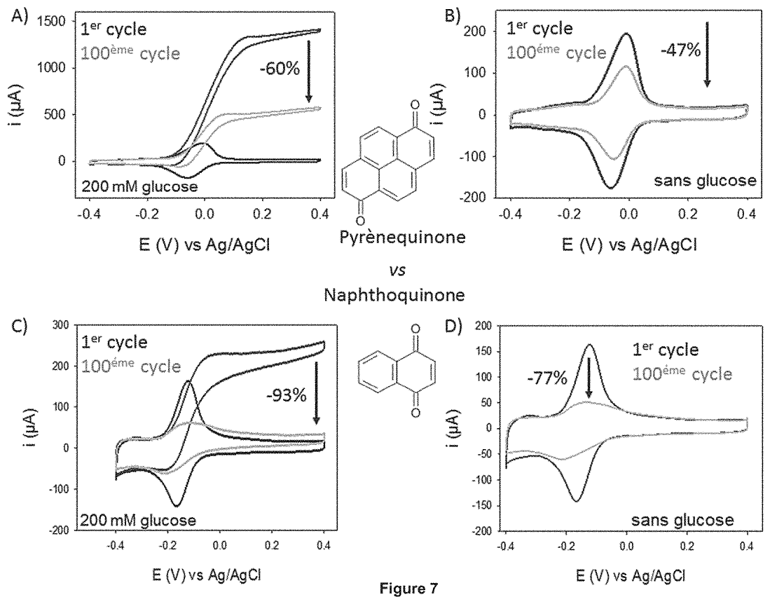

FIG. 7: Comparison of pyrenedione with 1,4-naphthoquinone by CV in terms of efficiency and stability of electron transfer (catalytic streams, A and C) and in terms of stability of redox activity after 100 cycles (non-catalytic stream, B and D).

Production of a Bioelectrode According to the Invention

A commercial 0.071 cm² glassy carbon electrode (sold by Bio-Logic, France) is modified by the addition of carbon nanotubes (suspension of 5 mg·mL⁻¹ in carbon nanotubes).

This suspension is made by adding 10 mg of unfunctionalized multi-wall carbon nanotubes (MWCNT Nanocyl™, 97%) in 2 mL of NMP (N-methyl-2-pyrrolidone). The dispersion is placed under ultrasonic stirring for 2 hours. 20 μL of this previously stirred MWCNT suspension is then deposited on the surface of the glassy carbon electrode.

The electrode is then placed under vacuum in a desiccator. The electrode is then removed from the desiccator when the solvent has evaporated and the carbon nanotubes are dry (on average a few hours, generally from 3 to 5 hours).

Functionalization of Electrodes Via Dropcasting with Pyrene

After functionalization of the electrode with carbon nanotubes, it is modified by adding 20 μL of a 10 mM-concentrated solution of pyrene dissolved in dichloromethane (conc. 5 mg/mL). The solvent is then evaporated at atmospheric pressure (approx. 100 kPa) and ambient temperature (approx. 25° C.).

Electrosynthesis of the Electrode by Chronoamperometry and Cyclic Voltammetry

The electrode modified with pyrene is placed in an electrolytic solution (phosphate buffer 0.2 M Na₂HPO₄ and 0.2 M NaH₂PO₄ of pH 7) degassed beforehand under argon. The electrode is then subjected by chronoamperometry to a current of 1 V using as a counter electrode a platinum electrode and a reference electrode of the Ag/AgCl type for 30 seconds. The electrode is then rinsed with distilled water to remove all traces of electrolyte carrier or organic molecules.

It should be noted that the activation of pyrene was also carried out by successive cyclic voltammetry scans ranging from −0.4 V to 1 V vs. Ag/AgCl. The number of cycles varies from 3 to 20 and the electrode is then rinsed with distilled water to remove all traces of electrolyte carrier or organic molecules. The results presented below were generally carried out using the electrode obtained by chronoamperometry but similar results were obtained by cyclic voltammetry (for example FIG. 1 (right)), and these two electrodes are considered to be of almost identical structure and performance.

Functionalization of the Electrode Via Dropcasting of the Biocompound

The FAD-GDH used in this example is a FAD-GDH from Aspergillus sp. (SEKISUI DIAGNOSTICS, Lexington, Mass., Catalog No. GLDE-70-1192) which has the following characteristics:

Appearance: lyophilized yellow powder.

Activity: >900 U/mg powder 37° C.

Solubility: readily dissolves in water at a concentration of: 10 mg/mL.

One activity unit: the amount of enzyme that will convert one micromole of glucose per minute at 37° C.

Molecular weight (gel filtration) 130 kD.

Molecular weight (SDS-PAGE): diffuse band at 97 kD indicative of a glycosylated protein.

Isoelectric point: 4.4.

Kₘ value: 5·10⁻² M (D-glucose).

This enzyme is specific. Sugars other than D-glucose have been tested at a concentration of 30 mM. 2-deoxy-D-glucose exhibits only 25% activity compared to that of D-glucose.

D-xylose exhibits 11%, D-galactose 0.7%, D-mannose 0.4%, D-trehalose 0.2% and D-fructose 0.1%, activity compared to that of D-glucose. L-glucose, D-mannitol, D-lactose, D-sorbitol, D-ribose, D-maltose and D-sucrose each exhibit less than 0.1% activity compared to that of D-glucose.

Beforehand, a 5 mg·mL⁻¹ solution of FAD-GDH is prepared in a buffer solution (phosphate buffer 0.2 M Na₂HPO₄ and 0.2 NaH₂PO₄ pH 7) and stored at −20° C. Before each deposit, the solution is removed from the freezer and defrosted. 20 μL of this solution is deposited by dropcasting on the modified electrode. The solvent is then evaporated at atmospheric pressure (approx. 100 kPa) and ambient temperature (approx. 25° C.).

Characterization of the Bioelectrode

The bioanode obtained is used in a standard electrolytic cell (with a platinum counter electrode and a reference electrode of the Ag/AgCl type) to constitute a cell when positioned in a glucose-concentrated medium. This cell is studied below and has the following characteristics:

Electrochemical Characterization

1. Electrosynthesis

FIG. 1 (left) shows the electrochemical response of a glassy carbon electrode coated with carbon nanotubes and pyrene. The black curve represents the electrochemical response of the electrode, only a capacitive current is observed corresponding to the contribution of the carbon nanotubes. The gray curve was recorded after having imposed a potential of 1 V for 30 seconds. A faradic signal is observed at a potential of −0.036 V vs. Ag/AgCl. The application of a potential of 1 V therefore induces the synthesis of a new species exhibiting redox properties.

The previous experiment was carried out by imposing a potential for a given time. It is also possible to electrogenerate the redox probe by successive scans. The different electrochemical cycles are shown in FIG. 1 (right), The black curve represents the first scan cycle and the gray curves represent subsequent cycles. During the first cycle, the absence of a redox signal at −0.05 V in the outward cycle is noticed. The redox peak appears on the return cycle. This behavior is similar to electropolymerization reactions.

Here it is not the formation of a redox polymer but the electrosynthesis of an electroactive system. At potentials close to 1 V, oxidation of the compound occurs, forming ketone bonds on the aromatic compounds which then become electroactive (Diagram 1). The molecules formed contain quinone functions giving them redox properties.

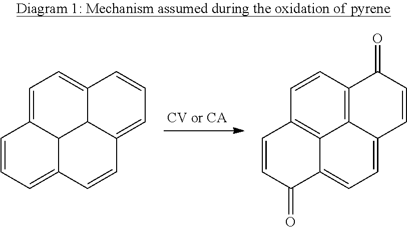

2. Characterization of the Redox Signal

The electrochemical response of the electrogenerated redox electrode is characteristic of a species immobilized at an electrode i.e. ΔE close to 0 mV (10 mV to 2 mV·s⁻¹) and the intensity of the oxidation and reduction peak is proportional to the scan rate (FIG. 2).

The nature of this product was also studied by varying the pH of the electrolytic solution (FIG. 3). The variation of the pH generates a modulation of the redox potential. The slope is −0.056, which is close to the theoretical value −0.059, indicating that this is a redox system involving the exchange of the same number of protons as electrons. It is very likely that this is an exchange of 2 electrons and 2 protons like many aromatic redox probes (naphthoquinone, anthraquinone, etc.). We can therefore assume that electrosynthesis generates the formation of ketone functions on the aromatic rings. In the case of this electrode which is functionalized with a pyrene unit, the assumed product formed is 1,6-pyrenedione or 1,4-pyrenedione. The electrogenerated redox couple is therefore pyrenedione/dihydroxypyrene with an exchange of 2 electrons and 2 protons. However, the literature (e.g. P. Barathi, A. Senthil Kumar, Langmuir, 29 (2013) 10617-10623, cited) differs on the exact nature of the compound formed and it is not necessarily possible to conclude on the number of ketone functions formed.

Study of the Catalytic Properties of Bioelectrodes

FIG. 4 shows the electrochemical response of the bioanode described above (MWCNT/pyrene redox/FAD-GDH) in the absence and presence of glucose. The black curve in the absence of glucose shows only the reversible electrochemical response of the immobilized redox probe. Conversely, in the presence of an aqueous solution of 200 mM glucose, a wave of oxidation is observed and characteristic of catalytic activity. Catalytic current occurs at the potential of the redox probe. This shows that the electrogenerated redox probe allows mediated electron transfer between the electrode and the FAD-GDH enzyme. The figure on the right shows the flow of current as increasing amounts of glucose are added. The maximum catalysis current of the range of 1.3 mA (6.5 mA·cm⁻²) is reached for glucose concentrations of 200 mM. This is also observed by the insert in the right drawing (4B) showing the evolution of the current as a function of the concentration which reaches a plateau for concentrations of 200 mM. The apparent Michaelis-Menten constant of the system is 39.8 mM.

Comparative Studies of Other Activated Polyaromatic Compounds

In order to establish the surprising properties of the enzyme electrode according to the invention, several comparative studies have been carried out using base materials other than pyrene. The bioanodes were produced in the same way and according to the same steps as for the activated pyrene electrode described above. The chosen activation method was cyclic voltammetry (FIGS. 5A and 5B) and chronoamperometry (FIG. 6) which generates the same behaviors. The only modification was the nature of the polycyclic compound.

Thus, FIG. 5 (left) shows the electrochemical response of the oxygenated derivative of anthracene after activation by cyclic voltammetry. The signature matches exactly that of anthraquinone, a commercial product. The drawing on the right shows the electrosynthesis of a perylene derivative under the same conditions. It is most likely a perylenequinone but such derivatives are not marketed which does not make it possible to determine the exact structure. The potential of these two components (˜−0.5 V for anthraquinone and ˜−0.2 for perylenequinone) does not allow electron transfer with FAD-GDH.

FIG. 6 shows the electrochemical response of the oxygenated derivative of phenanthrene after activation by cyclic voltammetry. This shows a transfer of electrons after electro-oxidation. The signature exactly matches that of phenanthraquinone, a commercial product. The catalytic current nevertheless remains low (a few tens of μA) compared to electro-oxidized pyrene (several hundred μA)

FIG. 7 shows the comparison of a pyrenedione electrode (according to the invention) with a 1,4-naphthoquinone electrode by cyclic voltammetry in terms of the efficiency and stability of electron transfer. In the context of the production of biofuel cells, it is necessary to avoid the release of the redox mediator in solution, which induces a decrease in performance over time as well as possible pollution in the case of the implantation of biofuel cells in living organisms. After 100 cycles of cyclic voltammetry of the electrode in the presence of 200 mM glucose, the catalytic current decreases by 60% for the pyrenedione derivative while it decreases by more than 93% in the case of the electrode functionalized by the 1,4-naphthoquinone unit (FIGS. 2A and C). A decrease of 47% and 77% of the non-catalytic faradic signal is observed respectively for pyrenedione and 1,4-napthoquinone (FIGS. 2B and D).

In the case of the pyrene unit, this has certain advantages for use in bioanodes as a redox mediator for FAD-GDH. The product is easily electrosynthesized and exhibits rapid electron transfer. The redox potential of the pyrene-quinone' pyrene-dihydroquinone couple has a potential close to the redox potential of the active site of the enzyme. In the presence of the FAD-GDH enzyme and glucose, a catalysis current is observed (FIG. 7A). In our example, the maximum catalytic current obtained for an MWCNT/pyrene-quinone IFAD-GDH electrode is 1.4 m. This catalytic wave appears at potentials close to the redox potential of FAD-GDH and therefore makes it possible to obtain high open-circuit voltages (OCV) in the case of the integration of this bioanode in a biofuel cell device. The OCV is a crucial parameter to obtain devices delivering high power.

1-10. (canceled)

11. A bioelectrode comprising a conductive material and having a surface on which are deposited carbon nanotubes, a redox mediator based on pyrene or a derivative thereof, oxidized in-situ, this oxidation forming ketone bonds on the aromatic ring of pyrene, and an enzyme capable of catalyzing the glucose oxidation.

12. The bioelectrode according to claim 1, wherein the enzyme is a flavin adenine dinucleotide-dependent glucose dehydrogenase or a flavin adenine dinucleotide-dependent glucose oxidase.

13. The bioelectrode according to claim 1, wherein the mediator is obtained by chronoamperometry and comprises the application, for a given time, of a potential of 1 V to the bioelectrode and to pyrene, or a derivative thereof, deposited in-situ on the surface of the bioelectrode.

14. The bioelectrode according to claim 3, wherein the given time preferably ranges from 30 seconds to 3 minutes.

15. The bioelectrode according to claim 1, wherein the mediator oxidized in-situ is obtained by cyclic voltammetry and comprises the application to pyrene, or a derivative thereof, deposited in-situ on the surface of the electrode, of a potential varying cyclically from −0.4 V to 1 V.

16. The bioelectrode according to claim 5, wherein a number of cycles varying from 3 to 20 is applied.

17. A process for producing a bioelectrode capable of glucose oxidation, the method comprising:
a) a step involving the oxidation of pyrene, or a derivative thereof, wherein the pyrene or the derivative is pre-deposited on the surface of a conductive material, a conductive material on the surface of which carbon nanotubes are also deposited, and
b) a step subsequent to step a), involving depositing an enzyme capable of catalyzing the glucose oxidation on the surface of the electrode.

18. The process according to claim 7, wherein the pyrene oxidation step is carried out by chronoamperometry and comprises applying a potential of 1 V at the surface for a given time.

19. The process according to claim 8, wherein the given time ranges from 30 seconds to 3 minutes.

20. The process according to claim 7, wherein the oxidation step is carried out by cyclic voltammetry and comprises applying a voltage varying cyclically from −0.4 V to 1 V at the surface.

21. The process according to claim 10, wherein a number of cycles is applied, the number varying from 3 to 20.

22. The process according claim 7, wherein the enzyme is a flavin adenine dinucleotide-dependent glucose dehydrogenase or a flavin adenine dinucleotide-dependent glucose oxidase.

23. A bioelectrode produced by the process described in claim 5.
